(19) United States
(12) Patent Application Publication
(10) Pub. No.: US 2011/0059029 A1
(43) Pub. Date: Mar. 10, 2011

(54) DUAL COMPONENT ORAL CARE PRODUCT

(75) Inventors: Rajnish Kohli, Hillsborough, NJ (US); Richard Scott Robinson, Belle Mead, NJ (US); Richard j. Sullivan, Atlantic Highlands, NJ (US); Diane Commins, Livingston, NJ (US); Nagaraja Jayaraman, Cos Cob, CT (US)

(73) Assignee: Colgate-Palmolive Company, New York, NY (US)

(21) Appl. No.: 12/866,639

(22) PCT Filed: Feb. 6, 2009
(86) PCT No.: PCT/US09/33295

§ 371 Date: Nov. 15, 2010

Related U.S. Application Data
(60) Provisional application No. 61/027,422, filed on Feb. 8, 2008.

(51) Int. Cl. A61K 8/21; A61K 8/19; A61K 8/24; A61Q 11/00

(52) U.S. Cl. 424/51; 424/49; 424/57

(57) ABSTRACT

A dental composition which, e.g., eliminates or substantially reduces the discomfort and pain associated with dentinal hypersensitivity and exhibits enhanced anticaries and remineralization benefits, which composition contains a first component containing a calcium source, a second component containing an anion source and at least one of the components containing a basic amino acid in free or salt form, and the first and second components being maintained separate from each other until dispensed and combined for application to teeth.

This application claims the benefit of U.S. Application Ser. No, 61/027,422 filed Feb. 8, 2008, the contents of which are incorporated herein by reference.

### FIELD OF THE INVENTION

The present invention relates to dual component dentifrice formulations, wherein reactive components in the formulation are sequestered from one another prior to use. In one embodiment, the invention relates to a desensitizing dentifrice composition which eliminates or reduces the discomfort and pain associated with dentinal hypersensitivity and more particularly to a desensitizing dental composition containing a basic amino acid in free or salt form and a calcium ion component and an anion component which exhibits unexpected enhanced anticavity and remineralization properties.

### BACKGROUND

Dentinal hypersensitivity is defined as acute, localized tooth pain in response to physical stimulation of the dentine surface as by thermal (hot or cold) osmotic, tactile combination of thermal, osmotic and tactile stimulation of the exposed dentin.

Exposure of the dentine, which is generally due to recession of the gums, or loss of enamel, frequently leads to hypersensitivity. The art has determined that dentine tubules open to the surface have a high correlation with dentine hypersensitivity, Abs, J. Clin. Periodontal. 14, 280-4 (1987). Dentinal tubules lead from the pulp to the cementum. When the surface cementum of the tooth root is eroded, the dentinal tubules become exposed to the external environment. The exposed dentinal tubules provide a pathway for transmission of fluid flow to the pulpal nerves, the transmission induced by changes in temperature, pressure and ionic gradients.

It is known to the art that potassium salts are effective in the treatment of dentinal hypersensitivity. For example, U.S. Pat. No. 3,863,006 discloses that toothpastes containing potassium salts such as potassium nitrate desensitize the teeth after tooth brushing for several weeks. It is believed by those skilled in the art that an elevation in the extracellular potassium concentration in the vicinity of pulpal nerves underlying sensitive dentin is responsible for the therapeutic desensitizing effect of topically applied oral products which contain potassium nitrate. Due to passive diffusion of potassium ion into and out of the open dentine tubules, repeated application of the active ingredient is necessary to build up the necessary concentration in the vicinity of the pulpal nerves.

It is believed that the improved pain relief is obtained from the use of potassium salts in combination with gradual mineralization on the dentin surface which can either totally or partially occlude dentin tubules. Total occlusion will dramatically reduce fluid flow within the tubules which stimulates pain. Partial occlusion of the dentin tubules is believed to increase delivery of potassium ion inside the tooth because the inward diffusive flux is less dependent upon tubule radius than outward fluid flow (due to positive pulpal pressures) (See D. H. Pashley and W G Mathews, Archs. Oral Biol. (1993) 38, 577-582). Therefore, this enhanced delivery of potassium should enhance relief.

It has also long been known to include fluoride releasing compounds in dentifrices as anticaries agents, and it has been established that these compounds are effective to reduce the incidence of dental caries. Fluoride compounds which are conventionally used are sodium fluoride, sodium monofluorophosphate and stannous fluoride. The fluoride compounds are effective mainly due to the fluoride ions which improve the acid resistance of tooth enamel and accelerate recalcification or remineralization of decayed teeth in their early stage when the demineralization has proceeded only slightly. By remineralization, pre-existing tooth decay and caries can be reduced or eliminated thereby reducing preexisting carious conditions in the tooth structure. The effect of improving the acid resistance of the enamel is believed to be due to the fact that the fluoride ions are incorporated into a crystal lattice of hydroxyapatite which is the main constituent of tooth enamel or, in other words, fluoride ions partially fluoridate hydroxyapatite and simultaneously repair the lattice irregularities.

The effectiveness of fluoride treatment is dependent upon the amount of fluoride ion which is available for deposition on the enamel being treated. It is, therefore, desirable to formulate dentifrice compositions which provide maximum fluoride ion availability in brushing solutions formed using the dentifrice.

Arginine and other basic amino acids have been proposed for use in oral care and are believed to have significant benefits in combating cavity formation and tooth sensitivity. Combining these basic amino acids with minerals having oral care benefits, e.g., fluoride and calcium, to form an oral care product having acceptable long term stability, however, has proven challenging. In particular, the basic amino acid may raise the pH and facilitate dissociation of calcium ions that can react with fluoride ions to form an insoluble precipitate. Moreover, the higher pH has the potential to cause irritation. At neutral pH or acidic pH, however, a system utilizing arginine bicarbonate (which the art teaches is preferred) may release carbon dioxide, leading to bloating and bursting of the containers. Moreover, it might be expected that lowering the pH to neutral or acidic conditions would reduce the efficacy of the formulation because the arginine may form an insoluble arginine-calcium complex that has a poorer affinity for the tooth surface, and moreover that lowering the pH would reduce any effect the formulation might have on buffering cariogenic lactic acid in the mouth. Partly because of these unaddressed formulation hurdles and partly because arginine has generally been viewed in the art as a potential alternative to fluoride rather than a co-active, there has been little motivation to make oral care products comprising both arginine and fluoride. Additional hurdles are potentially posed by addition of an antimicrobial agent. Commercially available arginine-based toothpaste, such as ProClude® and DenClude®, for example, contain arginine bicarbonate and calcium carbonate, but not fluoride nor any antimicrobial agent.

While the prior art discloses the use of various oral compositions for the treatment of dentinal hypersensitivity, dental caries, and enamel demineralization there is still a need for additional compositions and methods which provide improved performance in such treatments.

### SUMMARY OF THE INVENTION

In accordance with the present invention there is provided an oral composition and method for the treatment of dentinal hypersensitivity which exhibits improved anticavity and remineralization properties, the composition containing a calcium ion source component, anion source component, and at least one of the components containing basic amino acid, each component being optionally contained in an orally acceptable vehicle, the first and second components being maintained separate from each other until dispensed and combined for application to teeth requiring relief from dentine hypersensitivity whereby upon repeated application of the composition to the teeth increased relief from dentinal hypersensitivity is experienced by the user accompanied by improved resistance to cavities.

In another embodiment, the invention encompasses a method to improve oral health comprising applying an effective amount of the oral composition to the oral cavity of a subject in need thereof, e.g., a method to

a. reduce or inhibit formation of dental caries,
b. reduce, repair or inhibit early enamel lesions, e.g., as detected by quantitative light-induced fluorescence (QLF) or electrical caries measurement (ECM),
c. reduce or inhibit demineralization and promote remineralization of the teeth,
d. reduce hypersensitivity of the teeth,
e. reduce or inhibit gingivitis,
f. promote healing of sores or cuts in the mouth,
g. reduce levels of acid producing bacteria,
h. to increase relative levels of arginolytic bacteria,
i. inhibit microbial biofilm formation in the oral cavity,
j. raise and/or maintain plaque pH at levels of at least about pH 5.5 following sugar challenge,
k. reduce plaque accumulation,
l. treat dry mouth,
m. enhance systemic health, including cardiovascular health, e.g. reducing potential for systemic infection via the oral tissues,
n. whiten teeth,
o. to reduce erosion of the teeth,
p. immunize the teeth against cariogenic bacteria, and/or
q. clean the teeth and oral cavity.

### DESCRIPTION OF THE PREFERRED EMBODIMENTS

“Fluoride ion source” is defined as a source of soluble fluoride or a fluoride that is not covalently bonded.

Anion source is defined as fluoride ion source, phosphate ion source, or mixtures thereof.

“Calcium source” is defined as a source of calcium that would react readily with a phosphate ion to precipitate CaPO₄ or a calcium that is reactive with fluoride to produce CaF₂ or mixtures of fluorinated calcium-phosphate salts.

“Phosphate ion source” is defined as a source of phosphate that is not covalently bonded.

The composition of the present invention is a dual component composition, comprised of a first dentifrice component comprising a calcium ion source, e.g., at a pH of about 5 to about 9.9, and a second dentifrice component comprising an anion source, e.g., buffered to maintain the pH at a substantially neutral pH level, e.g., about 6.5 to about 7 having a basic amino acid, in free or salt form, present in one or both of the first and second dentifrice components. The two components are preferably combined in approximately equal weight proportions, so that about one-half of the concentration of any particular ingredient within either component will be present when the components are combined and applied to the teeth, as by brushing. Both components are preferably formulated to have similar physical characteristics, so that the two components may be simultaneously delivered in the desired predetermined amounts by extrusion when separately housed in a multicompartmented tube or pump device.

In a dual component dentifrice of the present invention, the one dentifrice component is prepared having an alkaline pH and a composition otherwise similar to that of the other having a buffered neutral pH. The pH of the alkaline component is adjusted to a pH of about 8.5 to about 9.7 and preferably about 9 to about 9.5. The pH of the combined dentifrice components is in the range of about 7.5 to about 8.6 and preferably about 7.5 to about 8.5.

An alkaline agent such as an alkali metal compound including sodium hydroxide, potassium hydroxide, sodium bicarbonate, sodium carbonate, N-sodium silicate (a sodium silicate in 34.6% water available from PQ Corporation), basic amino acid, or bicarbonate of a basic amino acid, e.g. arginine bicarbonate, is incorporated in the alkaline pH dentifrice component of the dual component dentifrice in amounts in the range of about 0.5 to about 15% by weight, preferably about 1 to about 8% by weight and most preferably at about 1 to about 5% by weight of the component. Mixtures of the above alkali metal compounds may also be used. Sodium hydroxide is the preferred alkaline agent. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of about 7 or greater, e.g., at least about 8.

Accordingly, basic amino acids include, but are not limited to, arginine, lysine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, 1-arginine, or a salt thereof.

The compositions of the invention are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the basic amino acid is neutralized with acid, e.g., hydrochloric, phosphoric or carbonic acid, to form a salt or partial salt, prior to being formulated with calcium, fluoride or other reactive components.

In various embodiments, the basic amino acid is present in an amount of about 0.5 wt. % to about 20 wt. % of the total composition weight, about 1 wt. % to about 10 wt. % of the total composition weight, for example about 1.5 wt. %, 3.75 wt. %, 5 wt. or 7.5 wt. % of the total composition weight.

The humectant used in the preparation of the vehicle for the dentifrice composition of the present invention is generally a mixture of humectants, such as glycerol, sorbitol and a polyethylene glycol of molecular weight in the range of about 200 to about 1000, but other mixtures of humectants and single humectants may also be employed. The humectant content is in the range about of 10% to about 50% by weight and preferably about 20 to about 40% by weight of the dentifrice component. The water content is in the range of about 20 to about 50% by weight and preferably about 30 to about 40% by weight.

Thickeners used in the preparation of the dentifrice vehicle include organic and inorganic thickeners. Inorganic thickeners which may be included in the dentifrice components include amorphous silicas such as Zeodent 165 available from Huber Corporation, and Sylox 15 from W. R. Grace.

Organic thickeners of natural and synthetic gums and colloids may also be used to prepare the dentifrice components of the present invention. Examples of such thickeners are carrageenan (Irish moss), xanthan gum, sodium carboxymethyl cellulose, starch, polyvinylpyrrolidone, hydroxyethylpropylcellulose, hydroxybutyl methyl cellulose, hydroxypropyl methyl cellulose, and hydroxyethyl cellulose.

The inorganic thickener may be incorporated in the dentifrice p of the present invention at a concentration of about 0.5 to about 5% by weight and preferably about 1 to about 3% by weight. The organic thickener may be incorporated in the compositions of the present invention at a concentration of about 1 to about 3% by weight and preferably about 0.4 to about 1.5% by weight.

Surfactants may be incorporated in the dentifrice compositions to provide foaming properties. The surfactant is preferably anionic or nonionic in nature. Suitable examples of anionic surfactants are higher alkyl sulfates such as potassium or sodium lauryl sulfate which is preferred, higher fatty acid monoglyceride monosulfates, such as the salt of the monosulfated monoglyceride of hydrogenated coconut oil fatty acids, alkyl aryl sulfonates such as sodium dodecyl benzene sulfonate, higher fatty sulfoacetates, higher fatty acid esters of 1,2 dihydroxy propane sulfonate.

The surfactant agent is generally present in the dentifrice component composition of the present invention at a concentration of about 0.5 to about 10% by weight and preferably about 1 to about 5% by weight.

Abrasives may be incorporated in the dentifrice composition of the present invention and preferred abrasives are siliceous materials, such as silica. A preferred silica is a precipitated amorphous hydrated silica, such as Sorbosil AC-35, marketed by Crosfield Chemicals, or Zeodent 115 from Huber Company but other abrasives may also be employed, including hydroxyapatite, sodium metaphosphate, potassium metaphosphate, tricalcium phosphate, calcium phosphate dihydrate, anhydrous dicalcium phosphate, calcium pyrophosphate, magnesium orthophosphate, trimagnesium phosphate, calcium carbonate, sodium bicarbonate, alumina trihydrate, aluminum silicate, calcined alumina and bentonite.

The concentration of abrasive in the dentifrice composition of the present invention will normally be in the range of about 5 to about 40% by weight and preferably about 10 to about 25% by weight.

The source of desensitizing potassium ion is generally a water soluble potassium salt including potassium nitrate, potassium citrate, potassium chloride, potassium bicarbonate and potassium oxalate with potassium nitrate being preferred. The potassium salt is generally incorporated in one or more of the dentifrice components at a concentration of about 1 to about 20% by weight and preferably about 3 to about 10% by weight.

Levels of active ingredients will vary based on the nature of the delivery system and the particular active. For example, the basic amino acid may be present at levels from, e.g., about 0.1 to about 20 wt % (expressed as weight of free base), e.g., about 0.1 to about 3 wt % for a mouthrinse, about 1 to about 10 wt % for a consumer toothpaste or about 7 to about 20 wt % for a professional or prescription treatment product. Fluoride may be present at levels of, e.g., about 25 to about 25,000 ppm, for example about 25 to about 250 ppm for a mouthrinse, about 750 to about 2.000 ppm for a consumer toothpaste, or about 2,000 to about 25,000 ppm for a professional or prescription treatment product. Levels of antibacterial will vary similarly, with levels used in toothpaste being e.g., about 5 to about 15 times greater than used in mouthrinse. For example, a triclosan mouthrinse may contain, e.g., about 0.03 wt % triclosan while a triclosan toothpaste may contain about 0.3 wt % triclosan.

Pyrophosphate salts having anticalculus efficacy useful in the practice of the present invention include water soluble salts such as dialkali or tetraalkali metal pyrophosphate salts such as Na₄P₂O₇ (TSPP), K₄P₂O₇, Na₂K₂P₂O₇, Na₂H₂P₂O₇ and K₂H₂P₂O₇. Polyphosphate salts include the water soluble alkali metal tripolyphosphates such as sodium tripolyphosphate and potassium tripolyphosphate.

The pyrophosphate salts are incorporated in the dentifrice composition of the present invention at a concentration of about 0.5 to about 2% by weight, and preferably about 1.5 to about 2% by weight and the polyphosphate salts are incorporated in the dentifrice composition of the present invention at a concentration of about 1 to about by weight.

Colorants such as pigments and dyes may be used in the practice of the present invention. Pigments include nontoxic, water insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green #1 lake, FD&C Blue #2 lake, FD&C R&D #30 lake and FD&C #Yellow 15 lake. The pigments have a particle size in the range of about 5 to about 1000 microns, preferably about 250 to about 500 microns, and are present at a concentration of about 0.5 to about 3% by weight.

Dyes used in the practice of the present invention are generally food color additives presently certified under the Food Drug & Cosmetic Act for use in the food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium slat of 4-{[4-(N-ethyl-p-sulffobenzylamino)-phenyl]-(4-hydroxy-2-sulfoniu-mphenyl)-mewthylene}-[1-(N-ethyl-N-p-sulfobenzyl)-3,5-cyclohexadienimine]-, FD&C Blue No. 1 (disodium salt of dibenzyldiethyldiaminotriphenylcarbino-1 trisulfonic acid of indigotin) and mixtures thereof in various proportions. The concentration of the dye for the most effective result in the present invention is present in the dentifrice composition in an amount from about 0.0005 percent to about 2 percent of the total weight.

A striped dentifrice product may be obtained using the dual component dentifrice embodiment of the present invention, wherein colorants of contrasting colors are incorporated in each of the dentifrice components to be dispensed; the colorants being pharmacologically and physiologically non-toxic when used in the suggested amounts. Colorants used in the practice of the present invention include both the pigments and dyes discussed above.

Any suitable flavoring or sweetening material may also be incorporated in the dentifrice composition of the present invention. Examples of suitable flavoring constituents are flavoring oils, e.g., oils of spearmint, peppermint, wintergreen, sassafras, clove, sage, eucalyptus, marjoram, cinnamon lemon, and orange, and methyl salicylate. Suitable sweetening agents include sucrose, lactose, maltose, sorbitol, xylitol, sodium cyclamate, perillatine, and sodium saccharin. Suitably, flavor and sweetening agents may together comprise about 0.01% to about 5% or more of the preparations.

Antibacterial agents are non-cationic antibacterial agents based on phenolic and bisphenolic compounds, halogenated diphenyl ethers such as Triclosan, benzoate esters and carbanilides as well as cationic antibacterial agents such as chlorhexidine digluconate. Such antibacterial agents can be present in quantities of from about 0.03 to about 1% by weight of the particular component.

When noncationic antibacterial agents or antibacterial agents are included in any of the dentifrice components, there is also preferably included from about 0.05 to about 5% of an agent which enhances the delivery and retention of the agents to, and retention thereof on oral surfaces. Such agents useful in the present invention are disclosed in U.S. Pat. Nos. 5,188,821 and 5,192,531; and include synthetic anionic polymeric polycarboxylates, such as about 1:4 to about 4:1 copolymers of maleic anhydride or acid with another polymerizable ethylenically unsaturated monomer, preferably methyl vinyl ether/maleic anhydride having a molecular weight (M.W.) of about 30,000 to about 1,000,000, most preferably about 30,000 to about 800,000. These copolymers are available for example as Gantrez. e.g., AN 139 (M.W. 500,000), AN 119 (M.W. 250,000) and preferably S-97 Pharmaceutical Grade (M.W. 700,000) available from 1SP Technologies, Inc., Bound Brook, N.J. 08805. The enhancing agents when present are present in amounts of about 0.05 to about 3% by weight.

To prepare the dentifrice components of the present invention, generally the humectants, for example, propylene glycol, polyethylene glycol ingredients, are dispersed with any organic thickeners, sweetener, pigments such as titanium dioxide and any polyphosphates included as anti-calculus ingredients. Water is then added into this dispersion along with any antibacterial agent such as Triclosan, any antibacterial enhancing agent such as Gantrez and any anticalculus additional agents. In the first neutral pH component a fluoride ion source desensitizing agent and phosphate buffering agent is added. In the second component an ingredient to adjust the pH to an alkaline level is added, such as sodium hydroxide. These ingredients are mixed until a homogenous phase is obtained for each component. Thereafter inorganic thickener, silica abrasive, flavor and surfactant ingredients are added and the ingredients mixed at high speed under vacuum of from about 20 to about 100 mm of Hg. The resultant product, in the case of each component, is a homogeneous, semi-solid, extrudible paste product.

The dentifrice composition may be applied to hypersensitive tooth surfaces in the form of a paste or gel by tooth brushing or topically applied by being painted directly the tooth surfaces in the form of a liquid varnish using a soft applicator brush.

Levels of active ingredients will vary based on the nature of the delivery system and the particular active. For example, the basic amino acid may be present at levels from, e.g., about 0.1 to about 20 wt % (expressed as weight of free base), e.g., about 0.1 to about 3 wt % for a mouthrinse, about 1 to about 10 wt % for a consumer toothpaste or about 7 to about 20 wt % for a professional or prescription treatment product. Fluoride may be present at levels of, e.g., about 25 to about 25,000 ppm, for example about 25 to about 250 ppm for a mouthrinse, about 750 to about 2,000 ppm for a consumer toothpaste, or about 2,000 to about 25,000 ppm for a professional or prescription treatment product. Levels of antibacterial will vary similarly, with levels used in toothpaste being e.g., about 5 to about 15 times greater than used in mouthrinse. For example, a triclosan mouthrinse may contain, e.g., about 0.03 wt % triclosan while a triclosan toothpaste may contain about 0.3 wt % triclosan.

Enhancing oral health also provides benefits in systemic health, as the oral tissues can be gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues. Providing a less acidic oral environment is also helpful in reducing gastric distress and creates an environment less favorable to Heliobacter, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health.

The multicomponent dentifrice composition embodiment of the present invention is packaged in a suitable dispensing container in which the components are maintained physically separated and from which the separated components may be dispensed synchronously as a combined ribbon for application to a toothbrush. Such containers are known in the art. An example of such a container is a two compartment dispensing container, such as a pump or a tube, having collapsible sidewalls, as disclosed U.S. Pat. Nos. 4,487,757 and 4,687,663; wherein, the tube body is formed from a collapsible plastic web such as polyethylene or polypropylene and is provided with a partition within the container body defining separate compartments in which the physically separated components are stored and from which they are dispensed through a suitable dispensing outlet.

Enhancing oral health also provides benefits in systemic health, as the oral tissues can be gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues. Providing a less acidic oral environment is also helpful in reducing gastric distress and creates an environment less favorable to Heliobacter, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### Example I

A two component (Component A and B) desensitizing dentifrice of the present invention was prepared, designated “Dentifrice X”, Component A and Component B. When combined in equal amounts for tooth brushing, Dentifrice X would be effective to provide enhanced anticavity and remineralization properties. The ingredients of Components A and B are listed in Table I below.

TABLE 1
Dentifrice X

Weight %
Component

| Ingredients | A | B |
|---|---|---|
| Deionized Water | 10.16 | 13.8 |
| Sodium Fluoride | 0.64 | 0 |
| Potassium Nitrate | 5 | 5 |
| Glycerin | 10 | 22 |
| Polyethylene glycol 600 | 3 | 0 |
| Xanthan gum | 0.7 | 0.7 |
| Carboxymethyl cellulose | 0.5 | 0.5 |
| Sorbitol 70% NC | 36 | 0 |
| Sodium saccharin | 0.4 | 0.4 |
| Titanium Dioxide | 1 | 0 |
| Dicalcium phosphate | 0 | 48 |
| L-Arginine | 5 | 5 |
| 80% Phosphoric acid | 1.8 | 1.8 |
| FD&C Blue #1 (1.25% | 0.3 | 0.3 |
| Zeodent 115 | 22 | 0 |
| Zeodent 165 | 1 | 0 |
| Sodium Lauryl Sulfate | 1.5 | 1.5 |
| Flavor | 1 | 1 |

In the preparation of Dentifrice X, components A and B are prepared wherein the glycerin, polyethylene glycol and organic thickeners are dispersed in a conventional mixer until the mixture becomes a slurry, which is smooth in appearance. Color and sweetener are dispersed in this slurry before the addition of water. L-arginine is then dispersed in the slurry and neutralized by the addition of phosphoric acid. The potassium nitrate is also added. Following mixture of these components, the silica, dicalcium phosphate, and sorbitol components are then added to the individual components, which are then mixed thoroughly. The fluoride, sodium lauryl sulfate, flavors and pigments are finally added to the individual dentifrice components which is followed by mixing another 5-15 minutes under vacuum to prepare the resultant component product.

The two components are packaged in a dual chamber tube to prevent reaction between the fluoride in A and the calcium in B. The dual chamber tube permits the two phases to be dispensed side by side as a striped toothpaste.

Providing fluoride as soluble sodium fluoride, together with high levels of calcium, phosphate, and arginine, results in a high availability of these ingredients at the tooth surface, where they are effective to reduce demineralization, promote remineralization, and repair damage to the enamel which leads to hypersensitivity and eventually cavitation of the teeth.

1. A dual component dental composition comprising
a first component comprising a calcium source,
a second component comprising an anion source,
wherein at least one of the components contains arginine, or a salt thereof,
the first and second components being maintained separate from each other until dispensed and combined.

2. The composition of claim 1 wherein the anion source comprises a fluoride ion source, a phosphate ion source, or mixtures thereof.

3. The composition of claim 2 wherein the anion source is a fluoride ion source which delivers a fluoride ion concentration of about 25 to about 25,000 ppm.

4. The composition of claim 2 wherein the anion source is a fluoride ion source which comprises a fluoride releasable salt.

5. The composition of claim 2 wherein the anion source is a phosphate ion source which comprises a phosphate releasable salt.

6. The composition of claim 5 wherein the phosphate releasable salt is a sodium phosphate salt.

7. The composition of claim 1 wherein each component contains arginine.

8. A method for eliminating or reducing the discomfort and pain associated with dentinal hypersensitivity which comprises
preparing a first component comprising a calcium source and a second component comprising an anion source, wherein at least one of the components contains arginine or a salt thereof,
separately housing the first and second components,
dispensing and combining the first and second components and
applying the combined components to an oral cavity surface whereby the anions are allowed to react in the presence of the arginine or a salt thereof with the calcium ions to precipitate a calcium complex.

9. The method of claim 8 wherein each component contains arginine.

10. The method of claim 8 wherein the anion source comprises a fluoride ion source, a phosphate ion source, or mixtures thereof.

11. The method of claim 8 wherein the anion source is a fluoride ion source which delivers a fluoride ion concentration of about 250 to about 25,000 ppm.

12. The method of claim 10 wherein the anion source is a fluoride ion source which comprises a fluoride releasable salt.

13. The method of claim 10 wherein the anion source is a phosphate ion source which comprises a phosphate releasable salt.

14. The method of claim 13 wherein the phosphate releasable salt is a sodium phosphate salt.

15. A method to:
a. reduce or inhibit formation of dental caries,
b. reduce, repair or inhibit early enamel lesions,
c. reduce or inhibit demineralization and promote remineralization of the teeth,
d. reduce hypersensitivity of the teeth,
e. reduce or inhibit gingivitis,
f. promote healing of sores or cuts in the mouth,
g. reduce levels of acid producing bacteria,
h. increase relative levels of arginolytic bacteria,
i. inhibit microbial biofilm formation in the oral cavity,
j. raise and/or maintain plaque pH at levels of at least pH 5.5 following sugar challenge,
k. reduce plaque accumulation,
l. treat dry mouth,
m. whiten teeth,
n. promote systemic health, including cardiovascular health, e.g., by reducing potential for systemic infection via the oral tissues,
o. reduce erosion of the teeth,
p. immunize the teeth against cariogenic bacteria, and/or
q. clean the teeth and oral cavity;
comprising applying an effective amount of the oral composition of claim 1 to the oral cavity.

16. The composition of claim 2, wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and combinations thereof.

17. The composition of claim 1, wherein at least one of the first or second component further comprises potassium nitrate or potassium chloride.

18. The method of claim 10, wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, and combinations thereof.

19. The method of claim 10, wherein at least one of the first or second component further comprises potassium nitrate or potassium chloride.
